(19) United States
(12) Patent Application Publication
(10) Pub. No.: US 2016/0073926 A1
(43) Pub. Date: Mar. 17, 2016

(54) Apparatus and method for determining the position of a medical instrument

(71) Applicants: David Grodzki, Erlangen (DE); Annemarie Hausotte, Erlangen (DE); Björn Heismann, Erlangen (DE); Arne Hengerer, Mohrendorf (DE); Mark-Aleksi Keller-Reichenbecher, Sandhausen (DE); Sebastian Schmidt, Weisendorf (DE)

(72) Inventors: David Grodzki, Erlangen (DE); Annemarie Hausotte, Erlangen (DE); Björn Heismann, Erlangen (DE); Arne Hengerer, Mohrendorf (DE); Mark-Aleksi Keller-Reichenbecher, Sandhausen (DE); Sebastian Schmidt, Weisendorf (DE)

(21) Appl. No.: 14/854,649

(22) Filed: Sep. 15, 2015

(30) Foreign Application Priority Data
Sep. 15, 2014 (DE) 102014218445.2

(51) Int. Cl. A61B 5/06; A61B 5/00; A61B 5/055

(52) U.S. Cl. CPC A61B 5/062; A61B 5/055; A61B 5/7425

(57) ABSTRACT

An apparatus for determining a position of a medical instrument within a patient receiving zone of a magnetic resonance tomography device is provided. The apparatus includes a sensor and a computing and control device. The sensor is arranged on or in the medical instrument, includes at least one magnetic field sensor for obtaining measured values of a magnetic flux density, and may be connected to the computing and control device for data transfer purposes. The computing and control device is embodied to control magnetic fields of the magnetic resonance tomography device and to determine the position of the medical instrument. The measured values of the at least one magnetic field sensor and control signals for controlling the magnetic fields of the magnetic resonance tomography device are included in the determination of the position of the medical instrument.

This application claims the benefit of DE 10 2014 218 445.2, filed on Sep. 15, 2014, which is hereby incorporated by reference in its entirety.

### BACKGROUND

The present embodiments relate to an apparatus for determining the position of a medical instrument within a patient receiving zone of a magnetic resonance tomography device. The present embodiments also relate to a corresponding method for determining the position of a medical instrument within a patient receiving zone of a magnetic resonance tomography device.

Apparatuses and methods for determining the position of a medical instrument are well-known in specialist literature and are already used in clinical practice. This includes methods, for example, in which markers are attached to anatomically relevant points and are detected by sensors (e.g., commercially available optical tracking systems). Other methods that use x-rays for imaging purposes, such as computed tomography, CT, x-ray imaging or rotation angiography, reconstruct a position of a medical instrument with the aid of mapping the medical instrument in an image data record, which may also be spatial.

A field of application, in which the information relating to the position of a medical instrument is of huge importance, is what is known as brachytherapy. Brachytherapy is a minimally invasive method of irradiating a tumor (e.g., a prostate carcinoma, a cervical carcinoma, a mammary carcinoma, or a larynx carcinoma) by internal radiation therapy or radiation treatment in an immediate target region. One or a number of radiation sources are positioned in close proximity to the region to be irradiated. One significant advantage over external beam radiotherapy, EBRT, is if radioisotopes with a correspondingly short range are selected (e.g., with beta emitters), the radiation exposure for the surrounding tissue is minimal, whereas with external beam radiotherapy, healthy tissue also is to be penetrated in order to reach the target.

In order to introduce the radiation sources, applicators or guides (e.g., catheter-type apparatuses or hollow needles) are frequently inserted or implanted into the body close to the tumor or directly into the tumor tissue. With the temporary brachytherapy, the radiation sources may survive in the body temporarily (e.g., for a few minutes or hours), or in the case of permanent brachytherapy may survive in the body for a longer or unlimited period of time. With permanent brachytherapy, reference may also be made to low dose rate brachytherapy, LDR. With temporary brachytherapy, since a more powerful radiation source is used to irradiate the tumor, reference may be made to high dose rate brachytherapy, HDR.

In order to determine the precise target position of the radiation source, a computed tomography (CT) or magnetic resonance tomography (MRT) recording of the region to be irradiated may be produced, for example, prior to the therapy. The precise dose distribution in the target region is calculated on an irradiation planning system with the aid of this data record. The number and the positions of the applicators to be introduced and the radiation sources are determined based on the ideal dose distribution on or in the tumor. On account of the dose planning, the radiation is only applied with a high dose where the tumor is located. A dose distribution may also take place after implantation of the applicators and if necessary once again during the insertion of the radiation sources for quality control purposes. As a result, the surrounding and in part most radiation-sensitive tissue is not unnecessarily irradiated, and damage is minimized. Contrary to an external irradiation, the skin is not damaged since irradiation is performed from the inside.

The actual brachytherapy is performed following a preliminary examination, the dose planning, and the acquisition of necessary materials. The patient is sedated or anesthetized in a sterile environment (OP), and the applicators are implanted. This may take place using 2D fluoroscopy. After successful control of the position of the applicators, the internal irradiation takes place with the aid of radioactive radiation sources (e.g., seeds; in the form of approximately one to five millimeter long capsules made of cesium-137). With the afterloading method, the seeds are inserted manually or automatically through the applicators into their target region and, if necessary, in stages. The radiation dose in the target region is calculated by way of the radiation intensity of the individual seeds to be expected and the dwell time of the individual seeds in the applicator or in the target region. If the forecast dwell time is reached, the seeds and the applicators are if necessary removed again in stages in the case of a temporary brachytherapy. The dwell time and the calculated applied dose may be documented.

Precise knowledge of the position of the applicators or the seeds is required for a dose calculation. A precise representation of the tumor and the surrounding organs at risk (OAR) is also important to be able to calculate a dose distribution both for the tumor volume and also for the organs at risk. In general, different imaging methods may be used here. However, in most cases, computed tomography is used in current clinical practice since spatially-resolved 3D data records in which the applicators may be easily identified may be supplied therewith. The disadvantage of using computed tomography is that the target organs may only be delimited inadequately (e.g., in a pelvis minor). Magnetic resonance tomography would be suitable here, nevertheless with the disadvantage that applicators may now only be identified with difficulty. These are to be laboriously identified and segmented by a user (e.g., a physician) in order to be considerable in a planning system. This disadvantage is so significant that magnetic resonance tomography was previously barely used for this application. Other failings of the magnetic resonance tomography, which play an important role in the dosimetry for EBRT methods, such as distortion, determination of attenuation values of the tissue, skin limits outside of the imaging region of the device, conversely hardly play any role in the brachytherapy because the target volume is close to the isocenter of the MR device, only the direct environment of the tumor is to be considered, and deviations in the radiation absorption barely carry any authority on account of the minimal range. The magnetic resonance tomography would therefore be very well suited to carrying out dose calculations for the brachytherapy if the problem in terms of determining the position of the applicators were to be resolved.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an apparatus for determining a position of a medical instrument within a patient receiving zone of a magnetic resonance tomography device is provided. As another example, a corresponding method to determine the position of a medical instrument within a patient receiving zone of a magnetic resonance tomography device is provided.

In one embodiment, an apparatus for determining the position of a medical instrument within a patient receiving zone of a magnetic resonance tomography device is provided. The apparatus includes a sensor device and a computing and control device. The sensor device is arranged on or in the medical instrument. The apparatus also includes at least one magnetic field sensor for obtaining measured values of a magnetic flux density and may be connected to the computing and control device for data transfer purposes. The computing and control device is embodied to control magnetic fields of the magnetic resonance tomography device and to determine the position of the medical instrument. The measured values of the at least one magnetic field sensor and control signals for controlling the magnetic fields of the magnetic resonance tomography device are included in the position determination of the medical instrument.

An apparatus, with the aid of which the position of a medical instrument (e.g., a catheter) that is disposed within a patient receiving zone of a magnetic resonance tomography device may be determined, is provided. The apparatus includes a sensor device and a computing and control device. The sensor device is arranged on or in the medical instrument. The position of the medical instrument may be defined, for example, by the location at which the sensor device is arranged on the medical instrument. The sensor device includes at least one magnetic field sensor (e.g., sensor) that is configured to obtain measured values of a magnetic flux density present at the site of the at least one magnetic field sensor. The sensor device may be connected to the computing and control device for data transfer purposes (e.g., for transmitting measured values of the magnetic flux density). The computing and control device is embodied to control magnetic fields of the magnetic resonance tomography device and to determine the position of the medical instrument. In order to determine the position of the medical instrument, the measured values of the at least one magnetic field sensor and the control signals are used to control the magnetic fields of the magnetic resonance tomography device. This provides that the magnetic resonance tomography device is controlled by the computing and control device by corresponding control commands for generating a local magnetic field in the patient receiving zone. The sensor device measures the flux density of the local magnetic field using the at least one magnetic field sensor and routes the measured values to the computing and control device that calculates the position of the medical instrument.

The at least one magnetic field sensor may be embodied as a Hall sensor or as an XMR sensor or as magnetometer.

Magnetometers (e.g., teslameters or Gaussmeters) are used to measure magnetic flux densities and are in principle known. XMR sensors, from x-magnetoresistive, are thin layer sensors with a resistance that depends on the magnetic flux to which the XMR sensors are exposed. Hall sensors use the Hall effect to measure magnetic fields. Such components are currently available in miniaturized form and are suited to installation in a medical instrument.

In a development, the sensor device includes at least three magnetic field sensors. The at least three magnetic field sensors are configured to determine the strength and direction of the magnetic flux density in each spatial direction.

By combining three magnetic field sensors, each of which is aligned in a different spatial direction, the strength and the direction of the field may be determined at any point in time. The three magnetic field sensors may be aligned in pairs orthogonally to one another in order in each case to obtain as large measured values of the three magnetic field sensors as possible. A compact design may be achieved if the magnetic field sensors are arranged in a shared housing.

In a further embodiment, the computing and control device, aside from determining the position of the medical instrument within a patient receiving zone of a magnetic resonance tomography device, is also configured to determine the location of the medical instrument within the patient receiving zone of the magnetic resonance tomography device. The measured values of the at least one magnetic field sensor and control signals for controlling the magnetic fields of the magnetic resonance tomography device are included in the determination of the location of the medical instrument.

Aside from the position or the point in the zone, the position or orientation in the zone of the medical instrument may also be of interest, so that the point and orientation are determined. In one embodiment, a movement of the medical instrument is determined by repeatedly determining the position and location over time. In order to determine the location of the medical instrument, as well as determining the position of the medical instrument, the measured values of the at least one magnetic field sensor and the control signals for controlling the magnetic fields of the magnetic resonance tomography device are included.

The computing and control may be configured to generate a field gradient in each spatial direction using a magnetic resonance tomography gradient system, and the sensor device is configured to measure the respective magnetic flux density. The measured values of the at least one magnetic field sensor and the control signals for controlling the magnetic fields of the magnetic resonance tomography device are included in the position determination and/or in the determination of the location of the medical instrument.

Magnetic resonance tomography devices include a magnet unit with a gradient coil unit for generating magnetic field gradients. The generated magnetic field gradients are used for a spatial encoding during imaging. The gradient coil unit is controlled by a gradient control unit of the magnetic resonance tomography device. The magnet unit with the gradient coil unit, the gradient coil unit and the gradient control unit may be combined to form the magnetic resonance tomography gradient system. By switching a field gradient in each spatial direction, measuring the magnetic flux densities and taking the control signals for controlling the magnetic fields of the magnetic resonance tomography device into account, the position and/or location of the medical instrument may be determined.

The computing and control device may be configured to implement the position determination and/or the determination of the location of the medical instrument during an image recording with the magnetic resonance tomography device.

In this embodiment, the position determination and/or the determination of the location of the medical instrument are implemented during an MR image recording. If the same, similar or extended magnetic field gradients are used for the position determination and/or the location determination as for a spatial encoding during an imaging process, a time advantage results.

A further embodiment provides that a difference of measured values of the at least one magnetic field sensor in a B0 field and measured values of the at least one magnetic field sensor in a B0 field with field gradients in each spatial direction are included in the position determination of the medical instrument.

In an embodiment, the B0 field is measured without gradient fields, and in a second measurement, the B0 field plus the gradient fields on the three spatial axes are determined. The gradient fields are determined by differentiation. The position of the medical instrument or of the sensor device in the zone may be concluded directly from these three values for the three axes.

In an alternative embodiment, the sensor device includes at least three magnetic field sensors. The at least three magnetic field sensors are configured to determine the strength and direction of the magnetic flux density in each spatial direction, and the computing and control device is configured to determine the location of the medical instrument using measured values of the at least three magnetic field sensors in a B0 field.

The location or orientation of the medical instrument or the sensor device is determined from the absolute values of the three magnetic field sensors in the three spatial directions in the B0 field. The direction is then produced from the vector with the respective absolute values of the three spatial directions.

In one embodiment, the apparatus includes a data transfer device for the data transfer of measured values from the at least one magnetic field sensor to the computing and control device. The data transfer device is configured for a wireless data transfer, for a wire-bound data transfer, or for an optical data transfer.

The transmission device may be embodied as an interface with connector that transmits the measured values as data in a wire-bound manner via a data line, wirelessly via a radio system (e.g., by a standardized Bluetooth data transfer technology), or optically by way of an optical waveguide.

The data transfer means may be configured for a wireless data transfer. A radio frequency antenna unit of the magnetic resonance tomography device is also configured as a receive antenna for the wireless data transfer of measured values from the at least one magnetic field sensor to the computing and control device.

This feature represents a particularly space-saving solution in terms of data transmission, since a radio frequency antenna unit of the magnetic resonance tomography device in the form of coils, which is already present in a magnetic resonance tomography device, is also used as a receive antenna for the wireless data transfer of measured values from the at least one magnetic field sensor to the computing and control device. Techniques for the wireless transmission of signals within a magnetic resonance tomography device are known, for example, from EKG devices.

The data transfer device may be configured for a wire-bound data transfer, and the data transfer device includes an interface. The interface is arranged on the medical instrument and is configured to control or use a further function of the medical instrument.

A double usage of the interface of the data transfer device includes a data transfer of the measured values from the at least one magnetic field sensor to the computing and control device and control of other functions of the medical instrument that enable the constructive outlay to be reduced. For example, the medical instrument may be a catheter that has pincers on a distal end that may be controlled via the interface of the data transfer device. Another exemplary embodiment is an applicator for brachytherapy, which may have an interface for an afterloader, which is disposed outside of the patient, so that the measured values may be used at this point in order to determine the position of an applicator. Since the afterloader is normally not connected during an MR recording, the computing and control device is connected to the end of the applicator. The interface may also be configured for the wire-bound data transfer and to receive or guide an instrument means (e.g., a wire with a radiator; also a mechanical interface).

The sensor device may be arranged on several positions in or on the medical instrument, and the sensor device includes at least one magnetic field sensor at each position in order to obtain measured values of a magnetic flux density. The computing and control device is configured to determine the position and/or location of the medical instrument.

With a rigid, rotationally symmetrical medical instrument or an instrument in which the position determination of just one position is of interest, a three-axle sensor on the tip of the applicator is already sufficient. With a flexible medical instrument, such as a catheter-type instrument, such as an applicator for a brachytherapy, a number of magnetic field sensors, which form the sensor device overall, may be attached in order to be able to determine the entire course of the medical instrument.

The computing and control device is favorably configured to register the position and location of the medical instrument in an image of the magnetic resonance tomography device and/or to adjust an image of the magnetic resonance tomography device as a function of the position and/or location of the medical instrument.

Since the apparatus may determine the position of a medical instrument within a patient receiving zone of a magnetic resonance tomography device, the precise location of the medical instrument relative to the gradient field is known. Since the coordinates of the MRT data records are likewise relative to the gradient field, these measurement results may be very easily overlayed with the MRT data records and recorded in a shared image. In a further aspect, the location of the medical instrument may be observed during a magnetic resonance tomography examination by the knowledge of the gradients switched during the MR examination being used. The time and the site at which magnetic field strength is to be expected are therefore known. The magnetic fields measured with the magnetic field sensors may therefore determine the precise location of the medical instrument in the position space at any time. This knowledge may be used to correct the movement of an MR image or to track the medical instrument (e.g., during insertion of an applicator/seed).

The computing and control device may include an image model of the medical instrument, and the computing and control device may be configured to superimpose the image model of the medical instrument in a positionally correct and/or location correct manner over an image of the magnetic resonance tomography device.

The image model of the medical instrument may be stored, for example, as a 3D data record, including the position of the sensor device within the medical instrument, in a database with further medical instruments or applicators and may then be selected. The image model or the data record may then be superimposed or faded in according to the MRT images.

In one embodiment, the medical instrument is an applicator for implementing a brachytherapy.

As apparent from the preceding embodiments, the described apparatuses are suited, for example, for determining the position of an applicator in order to implement a brachytherapy within a patient receiving zone of a magnetic resonance tomography device.

The computing and control device is embodied to transmit the position and/or location of the medical instrument to a brachytherapy planning system.

If the position and/or location of an applicator for implementing a brachytherapy within an examination object (e.g., a human patient) are available to a brachytherapy planning system, the planning or the implementation of a brachytherapy may be implemented much more precisely. MRT image data records, which may be obtained with the magnetic resonance tomography device, and the information relating to the location of the applicators in a brachytherapy planning system may be transmitted and used to plan the therapy. A use for MR-assisted implantation is also possible. Similarly, a treatment in the magnetic resonance tomography device is possible.

A system for determining the position of a medical instrument within a patient receiving zone of a magnetic resonance tomography device may also be provided. The system includes one of the previously described inventive apparatuses to determine the position of a medical instrument within a patient receiving zone of a magnetic resonance tomography device and a magnetic resonance tomography device.

A method for determining the position of a medical instrument within a patient receiving zone of a magnetic resonance tomography device is also provided. The method uses one of the previously described apparatuses to determine the position of a medical instrument within a patient receiving zone of a magnetic resonance tomography device.

In this case, the method includes acts for the purpose of which components of the apparatus are configured. With a computing and control device, which is configured to register the position and the location of the medical instrument in an image of the magnetic resonance tomography device, a method act of a method may include registering the position and the location of the medical instrument in an image of the magnetic resonance tomography device using the computing and control device.

### BRIEF DESCRIPTION OF THE DRAWINGS

FIG. 1 shows a representation to describe a brachytherapy according to the prior art;

FIG. 2 shows a schematic and exemplary representation of a sensor device having three magnetic field sensors for use in one embodiment of an apparatus;

FIG. 3 shows a schematic and exemplary representation of a medical instrument, on which a sensor device is arranged; and

FIG. 4 shows a schematic and exemplary representation of an apparatus for determining the position of a medical instrument within a patient receiving zone of a magnetic resonance tomography device and a magnetic resonance tomography device.

### DETAILED DESCRIPTION

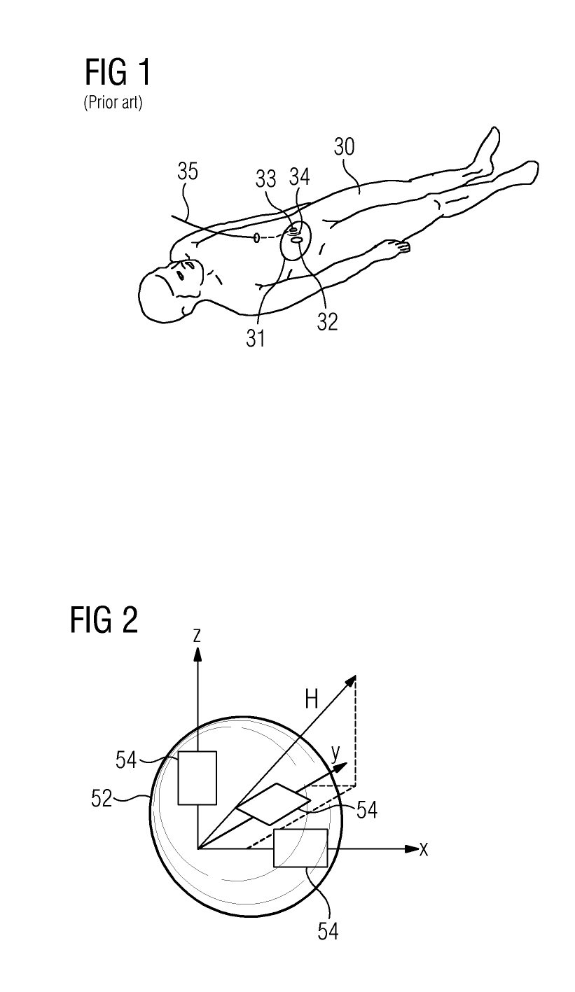

FIG. 1 shows a representation to describe a brachytherapy according to the prior art. With an examination object 30 (e.g., a human patient), a brachytherapy is implemented to treat a target object 32 (e.g., a tumor). An applicator 35 (e.g., in the form of a catheter) is inserted into a target region 31. The target region 31 includes at least the target object 32 (e.g., the target region is generally a volume within the examination object 30, within which at least the target object 32 lies). The applicator 35 allows a radiator 33 (e.g., a seed made of radionuclide; cesium-137, cobalt-60, iridium-192, iodine-125, palladium-103 or ruthenium-106) or also a miniaturized low energy x-ray emitter to be brought into the immediate vicinity of the target object 32. The radiator 33 emits high-energy beams, indicated in FIG. 1 by lines 34, that penetrate the target object 32. A significant advantage of brachytherapy is that the radiation effect relates to a very limited area around the radiation source. Consequently, tissue and organs in the environment of the radiator 33 are also irradiated so that the point of introduction, the type of radiator 33, and the duration of the treatment are to be very well considered in order to minimize the health risks for the examination object 30.

FIG. 2 shows a schematic and exemplary representation of a sensor device 52 having three magnetic field sensors 54 for use in an apparatus of one or more of the present embodiments. The magnetic field sensors 54 are aligned in each case in a different spatial direction (e.g., in pairs orthogonally to one another). The strength and direction of a magnetic field H may be determined at each point in time by this combination. The arrangement of the magnetic field sensors 54 in a shared housing indicated by a ball allows a compact design of the sensor means 52 to be achieved.

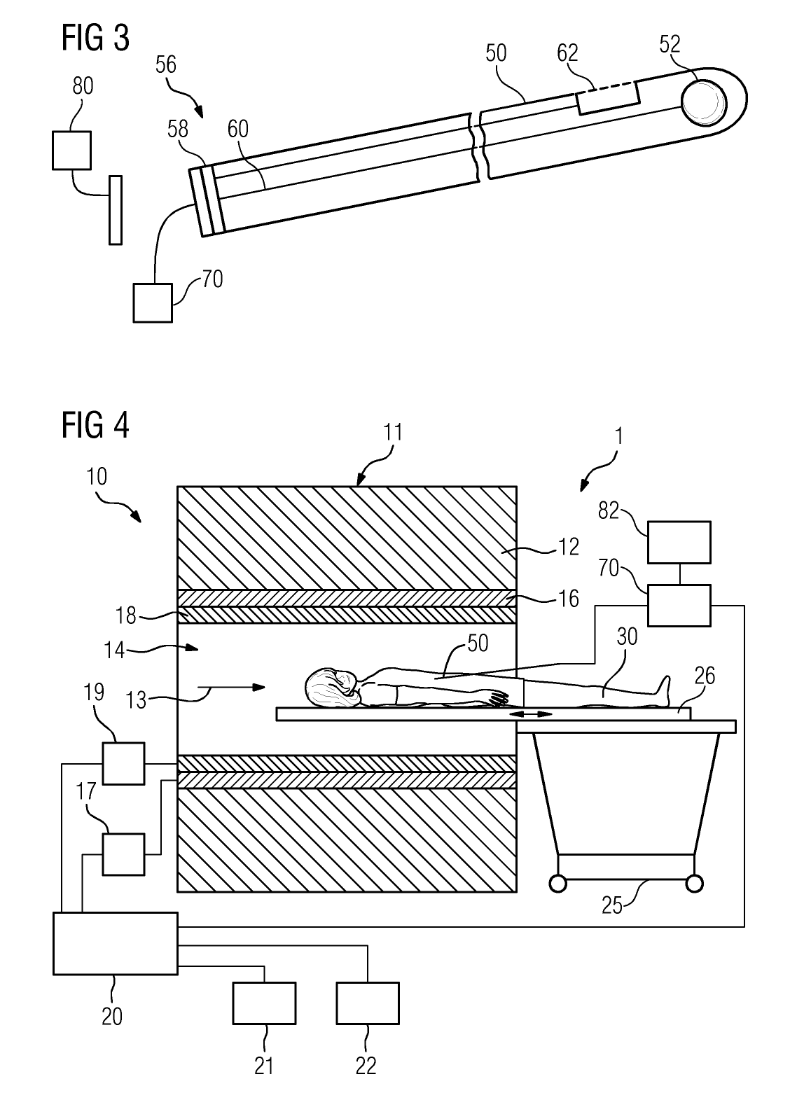

FIG. 3 shows a schematic and exemplary representation of a medical instrument 50 (e.g., an applicator for implementing brachytherapy). A sensor device 52 including three magnetic field sensors (not shown) is arranged on the distal end of the medical instrument 50. The sensor device 52 is configured to obtain measured values of a magnetic flux density within a patient receiving zone of a magnetic resonance tomography device and has a data transfer device 56 for the data transfer of measured values from the three magnetic field sensors to a computing and control device 70 (e.g., a computer). The data transfer device 56 in this exemplary embodiment includes metallic lines 60 for a wire-bound data transmission and an interface 58. The computing and control device 70 or a control facility 80 for transferring a radiator (e.g., an afterloader) may be connected to the interface 58. If the afterloader is connected, a radiator may be introduced into a region 62 by a wire (e.g., with a motorized adjustment device).

FIG. 4 shows a schematic and exemplary representation of an apparatus 1 for determining the position of a medical instrument 50 within a patient receiving zone 14 of a magnetic resonance tomography device 10 and a magnetic resonance tomography device 10. The magnetic resonance tomography device 10 includes a magnet unit 11 having a superconducting main magnet 12 for generating a powerful and, for example, constant main magnetic field 13, the B0 field. The magnetic resonance apparatus includes a patient receiving zone 14 for receiving an examination object 30 (e.g., a human patient). The patient receiving zone 14 is embodied in the present exemplary embodiment in a cylindrical design and is surrounded cylindrically in a peripheral direction by the magnet unit 11. An embodiment of the patient receiving zone 14 that deviates therefrom may, however, be provided. The examination object 30 may be pushed by a patient support apparatus 25 of the magnetic resonance device 10 into the patient receiving zone 14. The patient support apparatus 25 has a couch 26 that is configured to be movable within the patient receiving zone 14. The magnet unit 11 also has a gradient coil unit 16 for generating magnetic field gradients that are used for spatial encoding during imaging. The gradient coil unit 16 is controlled by a gradient control unit 17 of the magnetic resonance device 10. The magnet unit 11 also includes a radio frequency antenna unit 18 for exciting a polarization that becomes established in the main magnetic field 13 generated by the main magnet 12. The radio frequency antenna unit 18 is controlled by a radio frequency antenna control unit 19 of the magnetic resonance apparatus 10 and radiates radio frequency magnetic resonance sequences into an examination space that is substantially formed by a patient receiving zone 14 of the magnetic resonance tomography device 10. In order to control the main magnet 12, the gradient coil unit 17 and in order to control the radio frequency antenna control unit 19, the magnetic resonance apparatus includes a control unit 20. The control unit 20 centrally controls the magnetic resonance apparatus, such as performing a predetermined imaging gradient echo sequence, for example. The control unit 20 includes an evaluation unit (not shown in greater detail) for evaluating image data. Control information such as imaging parameters, for example, as well as reconstructed magnetic resonance images may be displayed on a display unit 21 (e.g., on at least one monitor) of the magnetic resonance tomography device 10 for viewing by an operator. The magnetic resonance tomography device 10 includes an input unit 22 by which information and/or parameters may be input by an operator during a measurement procedure. The magnetic resonance tomography device 10 disclosed may naturally include further components that magnetic resonance devices typically have. A general method of functioning of a magnetic resonance tomography device is also known to a person skilled in the art, so that a detailed description of the further components is not included. The apparatus 1 for determining the position of the medical instrument 50 (e.g., an applicator for performing a brachytherapy) within the patient receiving zone 14 of the magnetic resonance tomography device 10 includes a sensor device (not shown) and a computing and control device 70 (e.g., a computer). The sensor device is arranged on or in the medical instrument 50, includes at least one magnetic field sensor for obtaining measured values of a magnetic flux density, and is connected to the computing and control device 70 for data transfer purposes. The computing and control device 70 is embodied to control magnetic fields of the magnetic resonance tomography device 10 (e.g., by sending control signals to the control unit 20 of the magnetic resonance tomography device 10) and to determine the position of the medical instrument 50. The measured values of the at least one magnetic field sensor and control signals for controlling the magnetic fields of the magnetic resonance tomography device 10 are included in the position determination of the medical instrument 50. The computing and control device 70 may be embodied to control functions of the medical instrument 50 (e.g., by sending control signals) cause the medical instrument 50 to output a radiator. The computing and control device 70 is also configured to receive an image, which includes the medical instrument 50, from the magnetic resonance tomography device 10 and to register and/or superimpose the position and the location of the medical instrument 50 and in particular an image model of the medical instrument 50 in the image. The computing and control means 70 can also be integrated in the control unit 20 of the magnetic resonance tomography device 10. Furthermore, the computing and control means 70 can be configured to forward the position and location of the medical instrument 50 to a planning system 82 for brachytherapy. A combination comprising at least the apparatuses 1 for determining the position of a medical instrument 50 within a patient receiving zone 14 of a magnetic resonance tomography device 10 and the magnetic resonance tomography device 10 can also be referred to as a system for determining the position of a medical instrument 50 within a patient receiving zone 14 of a magnetic resonance tomography device 10.

In summary, further embodiments and advantages of the invention are described. The invention proposes inter alia an apparatus which allows an applicator for the implementation of a brachytherapy to be automatically located in a patient receiving zone of a magnetic resonance tomography device and the position of which is made clear for brachytherapy directly together with the anatomical recordings in a planning system.

To this end the brachytherapy applicator is equipped with a magnetic field sensor, which is used to define the position of the applicator in the magnetic field and to take this information into account within therapy planning. The advantage of this procedure is that non-linearities in the gradient system have a similar effect on the image data and also on the position data of the applicator so that the determined position of the applicator relative to the image data is always correct.

A simple, cost-effective and robust system is described in order to localize the brachytherapy applicators in the MRT in an unequivocal and motion-corrected manner.

The measurement can also be repeated at any time. Therefore the actual therapy can for instance also be performed in the magnetic resonance tomography device and in the process easily and quickly monitored at any time to determine whether the applicator is still located at the correct site. Or a gradient system, without a basic magnetic field, can be used to check the location during the therapy, in which the applicator can be located.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. An apparatus for determining a position of a medical instrument within a patient receiving zone of a magnetic resonance tomography device, the apparatus comprising:
a sensor; and
a computing and control device,
wherein the sensor is arranged on or in the medical instrument, includes at least one magnetic field sensor for obtaining measured values of a magnetic flux density, and is connectable to the computing and control device for data transfer,
wherein the computing and control device is configured to control magnetic fields of the magnetic resonance tomography device and to determine the position of the medical instrument, and
wherein the measured values of the at least one magnetic field sensor and control signals for controlling the magnetic fields of the magnetic resonance tomography device are included in the position determination of the medical instrument.

2. The apparatus of claim 1, wherein the at least one magnetic field sensor comprises a Hall sensor, an XMR sensor, or a magnetometer.

3. The apparatus of claim 1, wherein the sensor includes at least three magnetic field sensors configured to determine strength and direction of the magnetic flux density in each spatial direction.

4. The apparatus of claim 1, wherein the computing and control device, aside from determining the position of the medical instrument within the patient receiving zone of the magnetic resonance tomography device, is also configured to determine the location of the medical instrument within the patient receiving zone of the magnetic resonance tomography device, and
wherein the measured values of the at least one magnetic field sensor and control signals for controlling the magnetic fields of the magnetic resonance tomography device are included in the determination of the location of the medical instrument.

5. The apparatus of claim 1, wherein the computing and control device is configured to generate a field gradient in each spatial direction using a magnetic resonance tomography gradient system, wherein the sensor is configured to measure the respective magnetic flux density, and
wherein the measured values of the at least one magnetic field sensor and the control signals for controlling the magnetic fields of the magnetic resonance tomography device are included in the position determination, in the determination of the location of the medical instrument, or in a combination thereof.

6. The apparatus of claim 1, wherein the computing and control device is configured to implement the position determination, the determination of the location of the medical instrument, or a combination thereof during an image recording with the magnetic resonance tomography device.

7. The apparatus of claim 1, wherein a difference in the measured values of the at least one magnetic field sensor in a B0 field and measured values of the at least one magnetic field sensor in a B0 field with field gradients in each spatial direction are included in the position determination of the medical instrument.

8. The apparatus of claim 1, wherein the sensor includes at least three magnetic field sensors configured to determine strength and direction of the magnetic flux density in each spatial direction, and
wherein the computing and control device is configured to determine the location of the medical instrument using measured values of the at least three magnetic field sensors in a B0 field.

9. The apparatus of claim 1, further comprising a data transfer device for the data transfer of measured values from the at least one magnetic field sensor to the computing and control device,
wherein the data transfer device is configured for a wireless data transfer, a wire-bound data transfer, or an optical data transfer.

10. The apparatus of claim 9, wherein the data transfer device is configured for a wireless data transfer, and
wherein a radio frequency antenna unit of the magnetic resonance tomography device is also configured as a receive antenna for the wireless data transfer of measured values from the at least one magnetic field sensor to the computing and control device.

11. The apparatus of claim 9, wherein the data transfer device is configured for a wire-bound data transfer, and
wherein the data transfer device includes an interface arranged on the medical instrument and configured to control or use a further function of the medical instrument.

12. The apparatus of claim 1, wherein the sensor is arranged at a plurality of positions in or on the medical instrument,
wherein the sensor includes at least one magnetic field sensor at each position for obtaining measured values of a magnetic flux density, and
wherein the computing and control device is configured to determine the position, a location, or the position and the location of the medical instrument.

13. The apparatus of claim 1, wherein the computing and control device is configured to register the position and location of the medical instrument in an image of the magnetic resonance tomography device, adjust an image of the magnetic resonance tomography device as a function of the position, location, or position and location of the medical instrument.

14. The apparatus of claim 1, wherein the computing and control device includes an image model of the medical instrument, and
wherein the computing and control device is configured to superimpose the image model of the medical instrument in a positionally correct, location correct, or positionally correct and location correct manner over an image of the magnetic resonance tomography device.

15. The apparatus of claim 1, wherein the medical instrument is an applicator for implementing a brachytherapy.

16. The apparatus of claim 15, wherein the computing and control device is configured to transmit the position, location, or position and location of the medical instrument to a brachytherapy planning system.

17. A method comprising:
determining, using an apparatus, a position of a medical instrument within a patient receiving zone of a magnetic resonance tomography device, the apparatus comprising a sensor and a computing and control device, wherein the sensor is arranged on or in the medical instrument, includes at least one magnetic field sensor for obtaining measured values of a magnetic flux density, and is connectable to the computing and control device for data transfer, wherein the computing and control device is configured to control magnetic fields of the magnetic resonance tomography device and to determine the position of the medical instrument, and wherein the measured values of the at least one magnetic field sensor and control signals for controlling the magnetic fields of the magnetic resonance tomography device are included in the position determination of the medical instrument.
